(19) United States
(12) Patent Application Publication
(10) Pub. No.: US 2014/0141010 A1
(43) Pub. Date: May 22, 2014

(54) USE OF ADSL ACTIVATOR TO ACHIEVE GLYCEMIC CONTROL IN MAMMALS

(71) Applicant: Connexios Life Sciences Pvt. Ltd., Bangalore (IN)

(72) Inventors: Anup Mammen Oommen, Bangalore (IN); Swapnika Ramu, Bangalore (IN); Raghavendra Rao, Bangalore (IN)

(21) Appl. No.: 13/922,206

(22) Filed: Jun. 19, 2013

(30) Foreign Application Priority Data
Jun. 25, 2012 (IN) 2508/CHE/2012

(51) Int. Cl. A61K 39/395; A61K 45/06

(52) U.S. Cl. CPC A61K 39/3955; A61K 45/06
USPC 424/158.1

(57) ABSTRACT

The embodiments disclosed here in relates to use of activators of ADSL for the production of pharmaceutical agents to achieve glycemic control in mammals and which are therefore useful in the treatment of certain disorders that can be prevented or treated by activation of this enzyme.

### PRIORITY DETAILS

The present application claims priority from Indian Provisional Application Number 2508/CHE/2012, filed on 25 Jun. 2012, the disclosure of which is hereby incorporated by reference herein

### FIELD OF THE INVENTION

The embodiments disclosed herein relate to use of activators of ADSL for the production of pharmaceutical agents to achieve glycemic control in mammals and which are therefore useful in the treatment of certain disorders that can be prevented or treated by activation of this enzyme. In addition, it relates to a novel method for the reduction in the concentration of circulating glucose by activators of enzymes with similar or identical activity to the enzymatic activity of ADSL. It also relates to the treatment of conditions including but not limited to Type 1 Diabetes, non-insulin dependent type 2 diabetes mellitus (NIDDM), insulin resistance, obesity, impaired fasting glucose, impaired glucose and secondary complications caused due to the same.

### BACKGROUND OF THE INVENTION

Metabolic disorders, more specifically Type 2 Diabetes, obesity, cardiovascular diseases that results from both environmental and genetic factors are considered to be some of the fastest growing public health problems globally. These conditions may be associated with reduced insulin action and impaired glucose and lipid metabolism.

It is now well known that to approach a treatment mechanism for metabolic disorders, more particularly for achieving glycemic control, it is vital to regulate fatty acid and glucose homeostasis. Currently, to achieve glycemic control, AMP-activated protein kinase commonly referred to as AMPK, is one of the most sought after targets by many researchers of the world to achieve glucose control due to its role in regulation of whole body energy metabolism. AMPK is not only known to play a significant role in energy sensor by sensing intracellular AMP levels, but is also known to act as a regulator by being a crucial component in maintaining the energy balance within cells.

Under conditions of energy depletion, AMPK inhibits ATP-consuming pathways such as fatty acid synthesis, cholesterol synthesis and gluconeogenesis and stimulates ATP-generating processes such as fatty acid oxidation and glycolysis thus restoring the overall cellular energy homeostasis. Due to its dynamic nature, the inventors of this invention also believe that one of the most lucrative treatment regimes for glycemic control should involve activation of AMPK.

However, the inventors have realized that the biggest challenge in developing a small molecule that regulates AMPK is that AMPK has at least 12 known isoforms with different tissue specificities. Developing a small molecule which regulates one of the isoform will not give the desired effect as it may regulate AMPK in only some tissues and not all. On the flip side, a developing a small molecule that regulates all isoforms of AMPK at once would be very difficult due to the different conformations of the isoforms.

Furthermore, modulation of AMPK is less likely to provide robust control on cAMP mediated regulation of glycogenolysis from liver which is the primary driver under diabetic condition.

It is known that due to metabolic stress on the cell, the mitochondrial functions are adversely affected which inturn impacts the cellular ATP pools. The inventors of the invention found that, this in conjunction with reduced glucose metabolism due to insulin resistance, can result in reduced purine metabolites, making it rate limiting for further cellular processes.

Realizing that AMP/ATP ratio can act as an internal cellular sensor of energy state that can balance metabolic flux, the inventors of the invention found an alternate route to activate which would be instrumental in regulating glucose and fat metabolism. Hence to achieve these desirable effects, inventors using their proprietary network biology platform identified a novel mechanism by augmenting the purine denovo synthesis pathway that can increase cytosolic AMP level, activate AMPK (irrespective of the isoform specificities across tissues) as well as regulate cAMP by modulating Adenyl Cyclase enzyme which is involved in cAMP formation. The inventors found that activation of ADSL under pathological condition can increase the de novo route of purine synthesis thereby increasing the purine metabolites as well as AMPK activity providing a novel handle to manage glycemia.

### OBJECTS OF INVENTION

The principal object of the invention is to use ADSL activator to achieve glycemic control in mammals. Another object of the invention is to provide a method of prevention or treatment of a condition associated with ADSL activity in a mammal.

One other object of the invention is to provide a method for lowering elevated blood glucose level in mammals comprising administering at least one oral administration of a therapeutically effective amount of at least one ADSL activator.

### STATEMENT OF INVENTION

The first embodiment of the present invention is a method for lowering elevated blood glucose level in mammals comprising administering at least one oral administration of a therapeutically effective amount of at least one ADSL activator.

Another embodiment of the invention is a method where in the said at least one activator is administered in combination with at least one carrier substance.

In one other embodiment the said mammals demonstrate clinically inappropriate basal, fasting and post-prandial hyperglycemia.

Another embodiment of the invention is the use of administering a therapeutically effective amount of at least one ADSL activator to control elevated glucose level in mammals.

One other embodiment of the invention is the use of administering a therapeutically effective amount of at least one ADSL activator for treatment of diabetes and diabetes related complications.

Another embodiment of this invention is the method for lowering elevated blood glucose levels in mammals comprising administering at least one oral administration of a therapeutically effective amount of at least one activator of ADSL or of ADSL enzyme activity.

A further embodiment of the invention relates to a method of lowering elevated blood glucose levels in mammals comprising administering at least one oral administration of a therapeutically effective amount of at least one activator of ADSL or of ADSL enzyme activity in combination with an adjuvant selected from and not limited to (a) dipeptidyl peptidase-IV (DP-IV) inhibitors; (b) insulin sensitizing agents; (iv) biguanides; (c) insulin and insulin mimetics; (d) sulfonylureas and other insulin secretagogues; (e) alpha.-glucosidase inhibitors; and (f) GLP-1, GLP-1 analogs, and GLP-1 receptor agonists.

In another embodiment a preferred adjuvants are biguanides more preferably metformin.

### BRIEF DESCRIPTION OF DRAWINGS

FIG. 1 shows the decrease in ADSL mRNA level in rat primary hepatocytes under patho-physiological conditions

FIG. 2 shows the increased expression of mammalian overexpression vector for mouse ADSL in rat primary hepatocytes upon transfection

FIG. 3 shows increased AMPK phosphorylation caused due to ADSL overexpression under patho-physiological conditions.

FIG. 4 shows the ATP level in presence and absence of ADSL expression in primary hepatocytes under patho-physiological conditions.

FIG. 5 shows the decrease in hepatic TAG accumulation caused due to ADSL over expression under patho-physiological conditions.

FIGS. 6(A) and (B) shows the decrease in expression of inflammatory cytokines caused due to ADSL over expression under patho-physiological conditions.

FIG. 7 shows enhanced ability of insulin to repress gluconeogenesis caused due to ADSL overexpression under patho-physiological conditions.

FIG. 8 shows the fasting glucose level in high fat induced obese mice (C57B6 mice) in presence and absence of ADSL over expression

FIG. 9(A) gives the OGT (Oral glucose tolerance) following 2 g/kg oral glucose load in C57B6 mice fed on high fat diet for 12 weeks.

FIG. 9(B) shows the AUC profile (0-120 minutes) of high fat induced obese mice (C57B6 mice) delivered with ADSL gene compared with untreated control.

FIG. 10(A) gives the glucose level following 2 g/kg intra peritoneal load of pyruvate in C57B6 mice fed on high fat diet for 12 weeks.

FIG. 10(B) shows the AUC profile (0-60 minutes) of high fat induced obese mice (C57B6 mice) delivered with ADSL gene compared with the control

FIG. 11 shows SEQ ID 1 which is a nucleotide sequence used to overexpress ADSL gene in vivo. It is the open reading frame sequence of the ADSL gene.

### DETAILED DESCRIPTION OF THE INVENTION

The present invention aims at a new method to lower the level of blood glucose by agonizing the activity of Adenylosuccinate lyase (ADSL) which is an enzyme that in humans is encoded by the ADSL gene. Adenylosuccinate lyase converts adenylosuccinate to AMP and fumarate as part of the purine nucleotide cycle. ADSL catalyzes two reactions in the purine biosynthetic pathway that makes AMP; ADSL cleaves adenylosuccinate into AMP and fumarate, and cleaves SAICAR into AICAR and fumarate. Adenylosuccinate lyase is part of the β-elimination superfamily of enzymes and it proceeds through an E1cb reaction mechanism. The enzyme is a homotetramer with three domains in each monomer and four active sites per homotetramer.

ADSL is involved in both de novo synthesis of purines and formation of adenosine monophosphate from inosine monophosphate. It catalyzes two reactions in AMP biosynthesis: the removal of a fumarate from succinylaminoimidazole carboxamide (SAICA) ribotide to give aminoimidazole carboxamide ribotide (AICA) and removal of fumarate from adenylosuccinate to give AMP in pro-inflammatory signaling pathways.

The term “activation” or “agonization” shall mean increase in activity of the enzyme or protein unless specified particularly otherwise in this document.

The inventors through several experiments have shown that ADSL activation improves glycemic control and hence its use in treatment of diabetes, prediabetes and other diabetes related secondary complications is proved.

It is well known in prior art that, conditions such as steatosis, obesity and type 2 diabetes cause impairment of normal hepatic metabolism, resulting in the development of various pathological metabolic abnormalities which can collectively give rise to a state of insulin resistance. Normal metabolic homeostasis is maintained by several intracellular factors, most importantly AMP activated protein kinase (AMPK), a metabolic master switch known to regulate both glucose and fatty acid metabolism. AMPK acts as an energy sensor and is activated by energy depletion caused by low intracellular levels of adenosine triphosphate (ATP), as can be seen during fasting or exercise. Upon activation, AMPK serves to increase cellular energy levels by inhibiting anabolic (energy-consuming) pathways such as fatty acid and protein synthesis, and stimulating catabolic (energy-producing) pathways such as fatty acid oxidation, glucose transport and hepatic gluconeogenesis. Chronic exposure to metabolic overload, such as elevated levels of free fatty acids, inhibits AMPK, resulting in a loss of its energy-sensing capacity and consequent imbalance in metabolic homeostasis.

In addition to disturbed glucose and fatty acid metabolism, steatosis, obesity and T2DM are also known to be associated with an increase in the production of inflammatory cytokines from the liver, caused in part by the elevated levels of circulating free fatty acids and intra-hepatic lipid accumulation. The development of such chronic, low-grade inflammation during obesity is a known predisposing factor for the development of T2DM and associated complications.

Adenylosuccinate lyase (ADSL) is an enzyme that is part of the purine nucleotide cycle and that catalyzes two separate reactions resulting in the production of adenosine monophosphate (AMP) which can either activate AMPK or serve as a precursor for ATP synthesis. Given the central role of AMPK in maintaining metabolic homeostasis, the inventors of the invention believe that activation of ADSL is a novel route for mediating hepatic AMPK activation.

In the present invention, the ADSL activator or agonist can be any agent that activates ADSL enzyme activity including but not limited agonists of ADSL selected from small molecules, nucleotide sequences, polypeptide sequences and siRNA, pseudosubstrates that may cause activation of ADSL and binding proteins or antibodies against said enzyme proteins.

The invention concerns the use of ADSL activator for lowering of elevated glucose levels such as those found in mammals demonstrating clinically inappropriate basal, fasting and post-prandial hyperglycemia. The use according to the invention is more specifically characterized by the administration of ADSL activator in the prevention or alleviation of pathological abnormalities of metabolism of mammals such as hyperglycemia, low glucose tolerance, glucosuria, hyperlipidemia, hypertriglyceridemia, hypercholesterolemia, dyslipedemia, metabolic acidosis, obesity, diabetes mellitus and diabetes related secondary complications.

Accordingly compounds of the invention would be expected to have useful therapeutic properties especially in relation to prediabetic condition, insulin dependant diabetes mellitus, non insulin dependant diabetes mellitus and diabetic related secondary complications.

The term “Diabetic related secondary complication/s” shall include hyperlipidemia, hypertriglyceridemia, hypercholesterolemia, dyslipidemia, glaucoma, hypertension, atherosclerosis and its sequelae, retinopathy, nephropathy, neuropathy, osteoporosis, osteoarthritis, dementia, depression, neurodegenerative disease, psychiatric disorders, virus diseases, steatosis, fatty liver, nonalcoholic steatohepatitis, cirrhosis and inflammatory diseases

The term “therapeutically effective amount” or “effective amount” is an amount sufficient to effect beneficial or desired clinical results. An effective amount can be administered in one or more administrations. An effective amount is typically sufficient to palliate, ameliorate, stabilize, reverse, slow or delay the progression of the disease state.

The method according to the present invention is a new approach to the reduction of elevated circulating glucose concentrations in the blood of mammals.

The method is simple, commercially useful and appropriate for use in therapy, especially of human diseases, which are associated with elevated or inappropriate blood glucose levels.

The ADSL activators may be used or administered in combination with one or more additional drug(s) for the treatment of the disorder/diseases mentioned. The activating agent can be administered in the same formulation or in separate formulations. If administered in separate formulations the components can be administered sequentially or simultaneously with the other drug(s).

In addition to being able to be administered in combination with one or more additional drugs, the compounds of the invention may be used in a combination therapy. When this is done the component can be typically administered in combination with each other. Thus one or more therapeutic agents may be administered either simultaneously (as a combined preparation) or sequentially in order to achieve a desired effect. This is especially desirable where the therapeutic profile of each compound is different such that the combined effect of the two drugs provides an improved therapeutic result.

The ADSL activating agent may also be administered in combination with (or simultaneously or sequentially with) an adjuvant to increase compound performance. Suitable adjuvants may include (a) dipeptidyl peptidase-IV (DP-IV) inhibitors; (b) insulin sensitizing agents; (iv) biguanides; (c) insulin and insulin mimetics; (d) sulfonylureas and other insulin secretagogues; (e) alpha.-glucosidase inhibitors; and (f) GLP-1, GLP-1 analogs, and GLP-1 receptor agonists. The adjuvants may be part of the same composition, or the adjuvants may be administered separately (either simultaneously or sequentially). The order of the administration of the composition and the adjuvant will generally be known to the medical practitioner involved and may be varied.

In one other embodiment the ADSL activator is administered as a substitute to monotherapy or in combination with existing therapy regime. It may also be used in an event of failure of treatment by agent selected from the group consisting of dipeptidyl peptidase-IV (DPP-IV) inhibitors; (b) insulin sensitizing agents; (c) insulin and insulin mimetics; (d) sulfonylureas and other insulin secretagogues; (e) alpha.-glucosidase inhibitors; (f) GLP-1, GLP-1 analogs, and GLP-1 receptor agonists; and combinations thereof. Incase of such failure, the ADSL activators can be used instead of the failed therapy or in conjunction with the failed therapy to give the desired results.

Depending on the endogenous stability and on the bioavailability of the effectors, single or multiple administrations are suitable to reach the anticipated normalization of the blood glucose concentration.

### Experimental Data:

Through a series of experiments the inventors of this invention have established the role of ADSL Agonists in achieving glycemic control because of its impact on adipose tissue, muscle, pancreas, hepatocytes and macrophages. The impact of ADSL activation in vitro was studied using plasmid in which SEQ ID 1 was inserted into pCMV6-kan/neo vector for over expressing ADSL gene both in-vitro or in-vivo. Commercially available expression vector was obtained from origene

### ADSL Expression Levels Under Patho-Physiological Conditions

To study the ADSL expression in primary hepatocytes, the rat primary hepatocytes were subjected to metabolic overload and a combination of the inflammatory cytokines for 24 hours. RNA was then used for quantitative RT-PCR analysis to determine the expression levels of rat ADSL. The p-calues were calculated using Student's unpaired t-test.

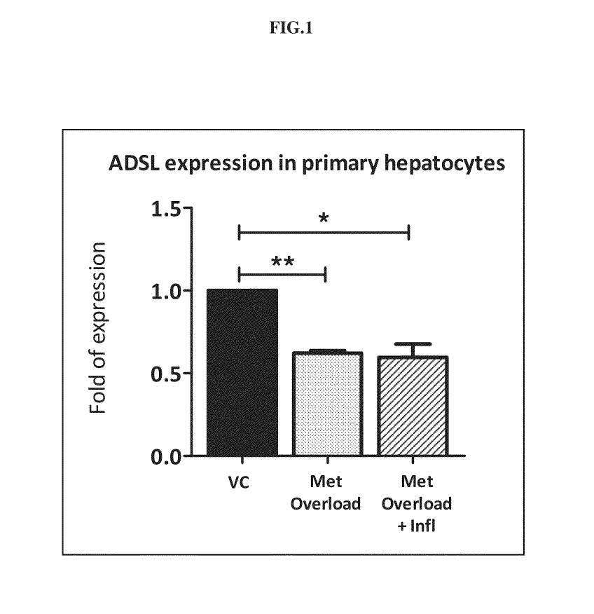

ADSL expression levels were found to be decreased in hepatocytes under patho-physiological conditions. As compared to untreated rat primary hepatocytes, cells treated with either metabolic overload or metabolic overload and a combination of inflammatory cytokines showed decreased levels of ADSL mRNA (FIG. 1), suggesting that such patho-physiological conditions may cause abnormalities in hepatic purine metabolism by decreasing ADSL activity.

### Overexpression of Mouse ADSL in Rat Primary Hepatocytes

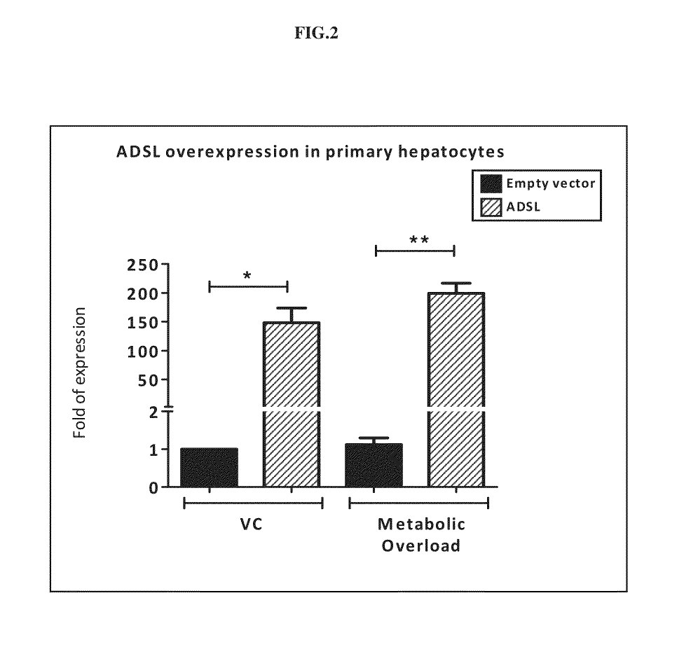

In order to increase expression levels and therefore activity of ADSL in rat primary hepatocytes, cells were transfected with a mammalian expression vector for mouse ADSL and the efficacy of the construct was assessed by measuring levels of mouse ADSL mRNA. For the purpose of this study, rat primary hepatocytes were transfected with either an empty control vector or a mammalian overexpression vector for mouse ADSL and subsequently either left untreated or treated with metabolic overload for 24 hours. RNA was then used for quantitative RT-PCR analysis to determine the expression levels of mouse ADSL. As seen in FIG. 2, transfected cells expressed significantly higher levels of ADSL, both under normal and patho-physiological conditions.

### Impact of ADSL Overexpression on AMPK Phosphorylation Under Patho-Physiological Conditions

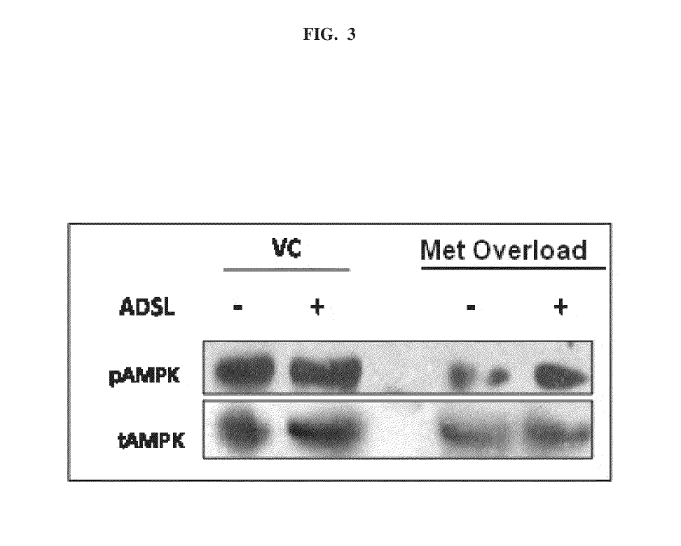

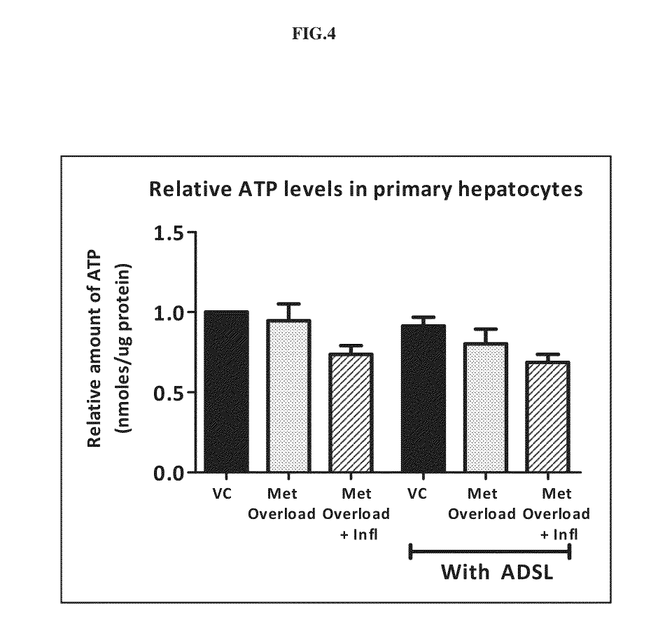

As described earlier, AMP generated by the enzymatic activity of ADSL can be used either as a precursor for ATP synthesis or directly to activate AMPK. Further, decreased AMPK activation as a result of chronic palmitate exposure has been demonstrated in various cell types. Therefore, in order to determine which route (ATP generation/AMPK activation) was favored as a result of ADSL overexpression, primary hepatocytes were transfected with ADSL overexpression construct and subsequently were left untreated or subjected to metabolic overload. Cell lysates were then used for Western blotting analysis of phospho-AMPK protein levels. As seen in FIG. 3, under normal conditions, ADSL overexpression did not appear to have any significant impact on AMPK phosphorylation. However, hepatocytes subjected to metabolic overload significantly decreased AMPK phosphorylation. Under these conditions, overexpression of ADSL resulted in enhanced AMPK phosphorylation. Thus, overexpression of ADSL may be able to exert beneficial effects under patho-physiological conditions via modulation of AMPK activity.

### Impact of ADSL Overexpression on Cellular ATP Levels Under Patho-Physiological Conditions

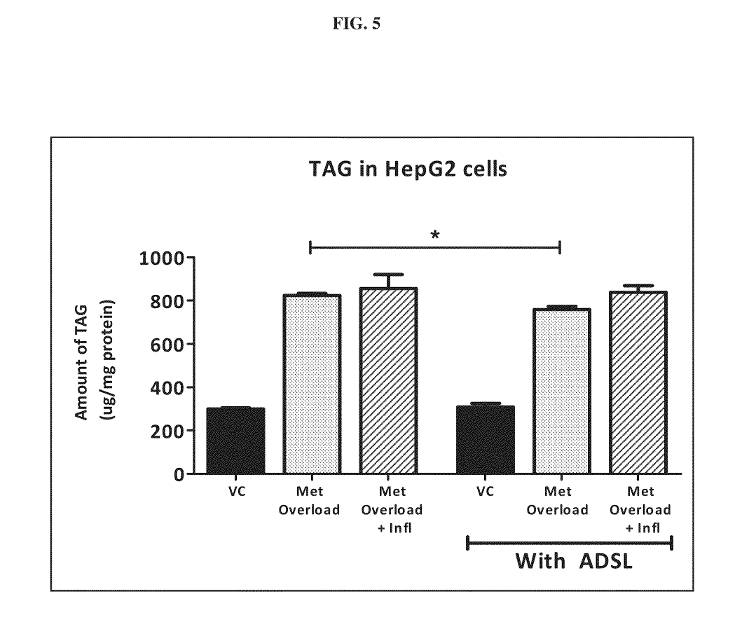

In order to confirm whether overexpression of ADSL also had any impact on cellular energy levels, ATP content was measured in primary hepatocytes under normal and disease conditions after ADSL transfection. It is well known that decreased hepatic ATP levels are associated with intracellular TAG accumulation and insulin resistance. As expected, upon treatment with metabolic overload and inflammatory cytokines, ATP levels in primary hepatocytes decreased (FIG. 4). However, the overexpression of ADSL under these conditions did not have any impact on cellular ATP levels. Therefore, it is likely that any impact of ADSL on hepatic metabolism is via AMPK activation rather than increased levels of cellular ATP.

### Impact of ADSL Overexpression on TAG Accumulation Under Patho-Physiological Conditions

One of the hallmarks of metabolic syndrome is the increased intrahepatic accumulation of triglycerides (TAG), which results in increased inflammation and insulin resistance. Through its impact on fatty acid oxidation, AMPK activation is known to decrease hepatic TAG levels. As expected, ADSL overexpression resulted in a small but significant decrease in intracellular TAG levels (FIG. 5) in human hepatoma HepG2 cells transfected with ADSL and then subjected to metabolic overload either alone or in combination with inflammatory cytokines. Thus, ADSL overexpression may promote improved lipid handling in the liver via fatty acid oxidation, thus decreasing intrahepatic lipid accumulation.

### Impact of ADSL Overexpression on Inflammatory Cytokine Gene Expression Under Patho-Physiological Conditions

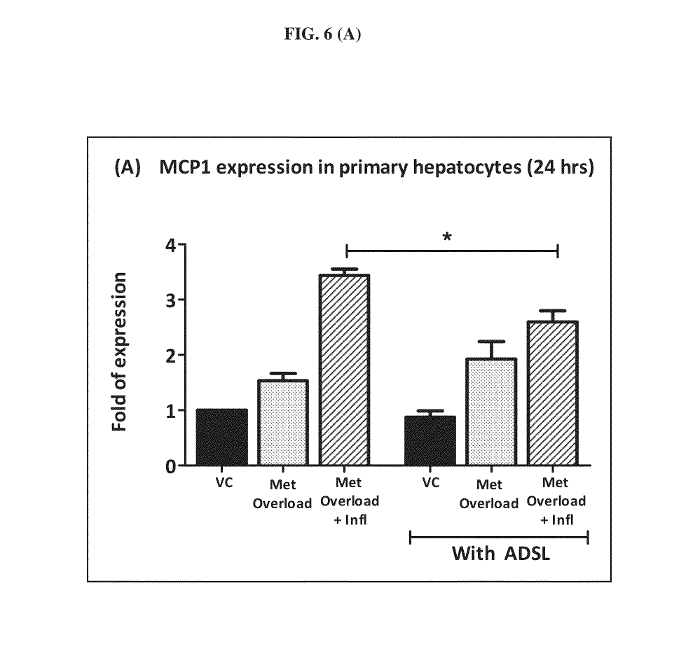

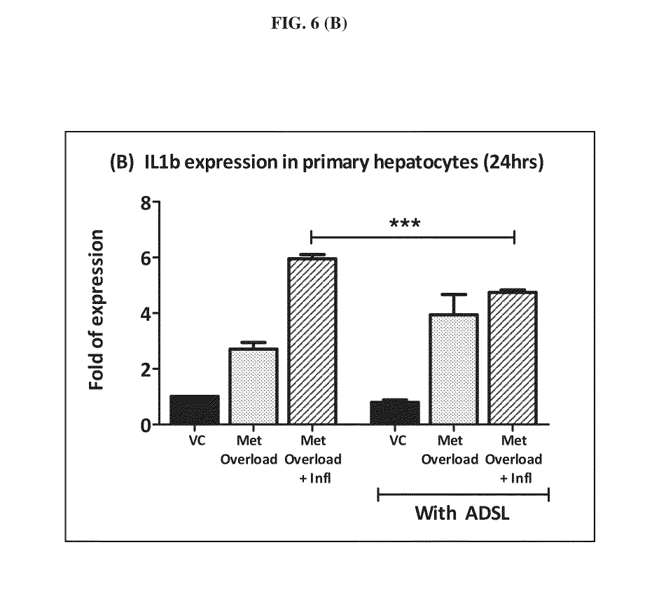

As described earlier, elevated levels of inflammatory cytokine production by the liver are associated with steatosis, obesity and T2DM. In order to determine whether ADSL activation could have a beneficial impact in reducing hepatic inflammation, primary hepatocytes were transfected with ADSL and then subjected to metabolic overload either alone or in combination with inflammatory cytokines. The expression levels of macrophage chemotactic protein-1 (MCP-1) and interleukin-1 beta (IL-1b), two well-characterized acute phase proteins, were measured and it was seen that ADSL overexpression was able to reduce the mRNA levels of both these cytokines under patho-physiological conditions. Thus, ADSL overexpression reduces the inflammatory stress created by metabolic overload and inflammation. FIG. 6 shows that ADSL overexpression decreases the expression of inflammatory cytokines under patho-physiological conditions.

### Impact of ADSL Overexpression on Hepatic Gluconeogenesis and Insulin Sensitivity Under Patho-Physiological Conditions

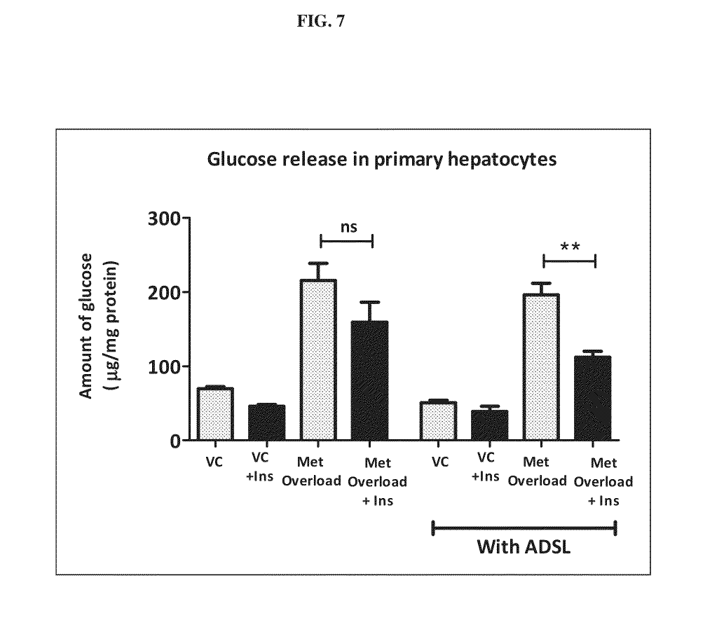

As discussed earlier, metabolic overload and increased inflammation result in the development of hepatic insulin resistance. As a result, the normal functions of insulin in modulating hepatic metabolism are lost. In the context of glucose handling, one of the key functions of the liver is to generate and release glucose during the fasting state for utilization by other tissues, a process known as gluconeogenesis. Upon feeding, elevated insulin levels serve to repress gluconeogenesis, thereby preventing excessive glucose excursion and hyperglycemia. Upon the development of insulin resistance, this control is lost and gluconeogenesis proceeds even in the presence of insulin, contributing to hyperglycemia. AMPK is also known to strongly repress hepatic gluconeogenesis. In order to assess whether ADSL overexpression could exert a beneficial effect on glucose control in the liver, primary hepatocytes were transfected with ADSL expression vector and then subjected to metabolic overload for 24 hours. Subsequently, metabolic overload was removed and the cells were exposed to glucose-free media containing gluconeogenic substrates either alone or with insulin, following which the amount of glucose released by the cells into the media was measured. As seen in FIG. 7, in the normal untransfected hepatocytes, insulin was able to repress gluconeogenesis. However, under patho-physiological conditions, addition of insulin was not able to significantly repress gluconeogenesis, suggesting the development of insulin resistance. Under these conditions, overexpression of ADSL was able to restore insulin-mediated repression of gluconeogenesis. Thus, ADSL may be able to increase hepatic insulin sensitivity, thereby resulting in modulation of gluconeogenesis.

### In-Vivo Experimental Data

C57B6 mice were fed on high fat diet for 12 weeks. The mice were randomized into two groups based on their oral glucose tolerance. The difference in the AUC 0-120 min between the groups was not statistically significant. To one group of mice ADSL gene was delivered to liver through tail vein injection. Control group mice received empty DNA vector which did not carry ADSL gene. In all the experiments presented below, students t-test (unpaired) was used for statistical analysis and p value<0.005 was considered statistically significant

### ADSL Overexpression Results in Fasting Glucose Control in DIO (Diet Induced Obese) Background:

To study the effect of ADSL agonism on fasting glucose, C57B6 mice were fed on high fat diet for 12 weeks. 20 micro grams of CMV-ADSL (or empty vector) was injected through tail vein. 21 days after this, mice from two groups were fasted for 12 hours followed by glucose estimation in the blood from tail tip.

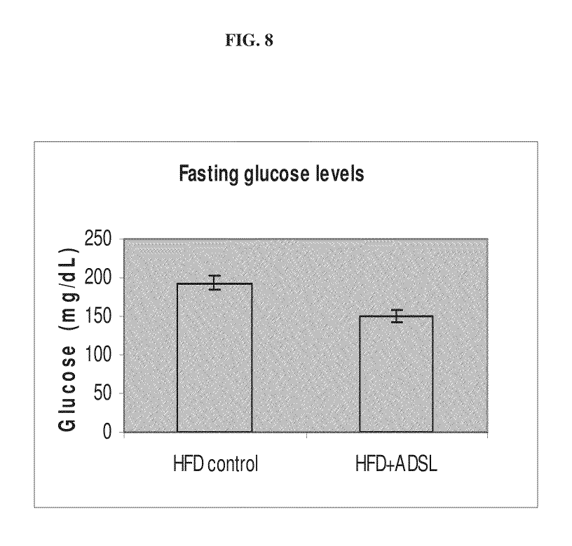

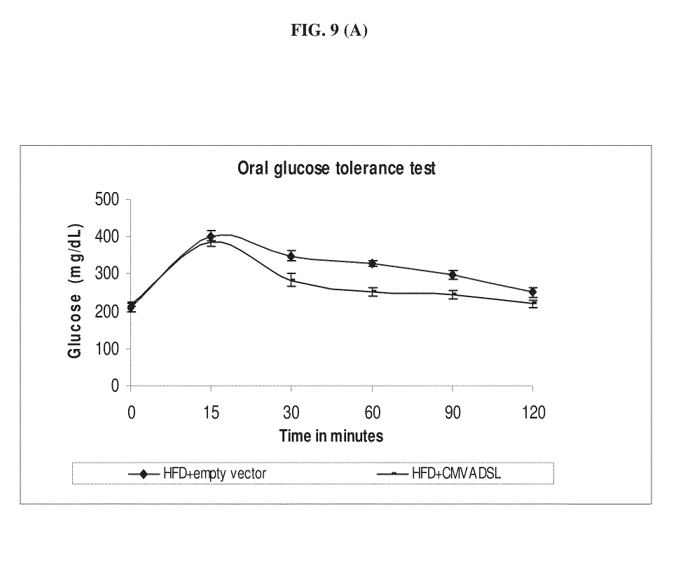

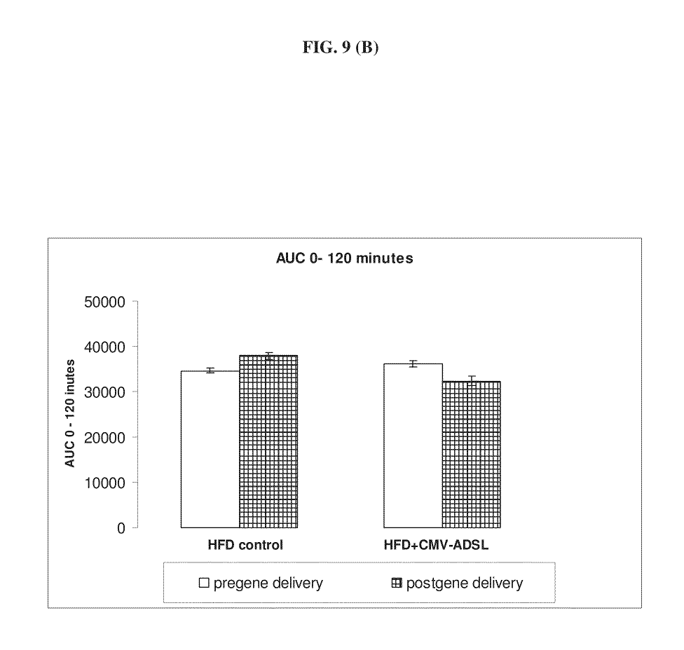

21 days following the gene delivery, there was a significant reduction in the fasting glucose levels in the test group of mice (those in which ADSL gene was introduced) compared to control mice in which gene was not delivered (FIG. 8). Initially (7 days post gene delivery), fasting glucose levels were not different between the control and gene-delivered mice (0 min, FIG. 9a). However, 21 days after gene delivery, as indicated in FIG. 8, the DIO (diet induced obese) mice group with ADSL overexpression in the liver showed substantially lowered fasting glucose compared to control mice in which ADSL was not introduced.

### ADSL Overexpression Results in Improved Glucose Tolerance

To study the effect of ADSL agonism on glucose tolerance, C57B6 mice were fed on high fat diet for 12 weeks. 20 micro grams of CMV-ADSL (or empty vector) was injected through tail vein. 7 days after this, mice from two groups were fasted for 6 hours followed by glucose estimation in the blood from tail tip (0 min reading). Glucose load was given at the dose of 2 g/kg, p.o. Subsequently glucose was estimated at various time points 15, 30, 60, 90, 120 minutes (FIG. 9a). The data was analyzed for the % change in the AUC following gene delivery (FIG. 9 b)

As indicated in FIG. 9 when ADSL was overexpressed in the liver, the DIO mice showed improved glucose tolerance. The ADSL treated mice showed 14% decrease in the AUC 0-120 min compared to age matched HFD control mice. ADSL gene expression resulted in 10% decrease in the AUC 0-120 min in the gene delivered mice when compared to the glucose tolerance data prior to gene delivery in the same mice.

### ADSL Overexpression Results in Reduced Gluconeogenesis:

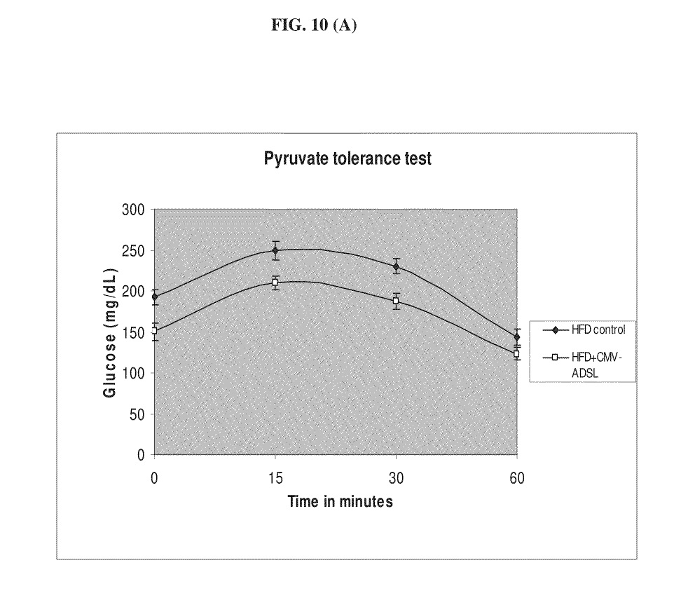

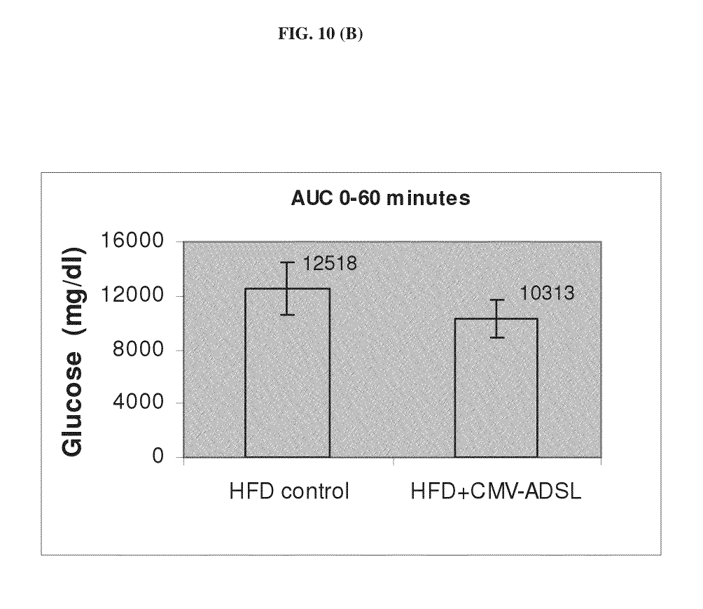

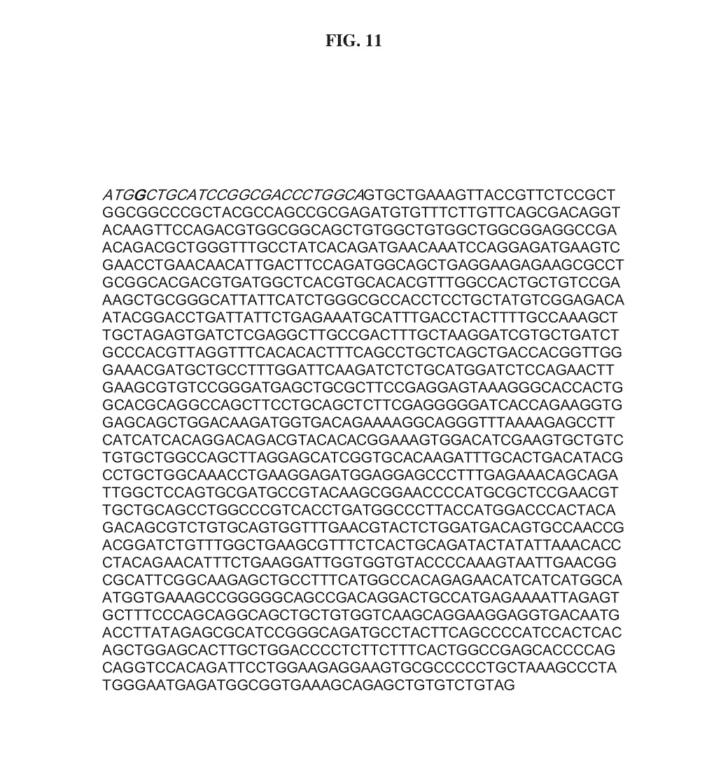

To study the effect of ADSL agonism on gluconeogenesis, C57B6 mice were fed on high fat diet for 12 weeks. 20 micro grams of CMV-ADSL (or empty vector) was injected through tail vein. 21 days after this, mice from two groups were fasted for 12 hours followed by glucose estimation in the blood from tail tip (0 min reading). Pyruvate load was given at the dose of 2 g/kg, i p. Subsequently glucose was estimated at various time points 15, 30, 60, 90, 120 minutes (FIG. 10a). The data was analyzed for the % change in the AUC following gene delivery (FIG. 10b)

Pyruvate serves as substrate for gluconeogenesis and results in the formation of glucose. Pyruvate tolerance is routinely used as an index of gluconeogenesis. We employed this test to evaluate the effect of ADSL overexpression on inhibition of gluconeogenesis. As indicated in the FIG. 10a the ADSL gene delivered mice showed decreased glucose output following pyruvate infusion. There was a 17% decrease in the AUC 0-60 min in the ADSL group compared to age matched control DIO mice (FIG. 10b).

We claim:

1. A method for lowering elevated blood glucose levels in mammals comprising administering a therapeutically effective amount of at least one activator of Adenylosuccinate lyase (ADSL) or ADSL like enzyme activity.

2. The method as claimed in claim 1 wherein said at least one activator is administered in combination with at least one carrier substance.

3. The method as claimed in claim 1 wherein said at least one activator is administered in multiple administrations.

4. The method according to claim 1 wherein the mammals demonstrate clinically inappropriate basal and postprandial hyperglycemia.

5. The method according to claim 1, wherein the administration is for the prevention or alleviation of pathological abnormalities of metabolism of mammals including but not limited to hyperglycemia, low glucose tolerance, glucosuria, hyperlipidemia, hypertriglyceridemia, hypercholesterolemia, dyslipedemia, metabolic acidosis, non alcoholic fatty liver diseases, cardiovascular diseases, diabetes mellitus and diabetes mellitus related secondary complications.

6. The method according to claim 1, wherein said at least one activator is administered in combination with an adjuvant selected from a group consisting of at least one dipeptidyl peptidase-IV inhibitor, at least one insulin sensitizing agent, at least one biguanide, insulin, at least one insulin mimetic, at least one insulin secretagogues, at least one alpha glucosidase inhibitors, GLP-1, at least one GLP-1 analog and at least one GLP-1 receptor agonist.

7. The method according to claim 6, wherein the said combination is administered simultaneously

8. The method according to claim 6, wherein the said combination is administered sequentially.

9. Use of activator of Adenylosuccinate lyase (ADSL) or ADSL like enzyme activity for lowering elevated blood glucose levels in mammals.

10. The use as claimed in claim 9 wherein said at least one activator is administered in combination with at least one carrier substance.

11. The use as claimed in claim 9 wherein said at least one activator is administered in multiple administrations.

12. The use according to claim 9 wherein the mammals demonstrate clinically inappropriate basal and postprandial hyperglycemia.

13. The use according to claim 9, wherein the said administration is for the prevention or alleviation of pathological abnormalities of metabolism of mammals including but not limited to hyperglycemia, low glucose tolerance, glucosuria, hyperlipidemia, hypertriglyceridemia, hypercholesterolemia, dyslipedemia, metabolic acidosis, non alcoholic fatty liver diseases, cardiovascular diseases, diabetes mellitus and diabetes mellitus related secondary complications.

14. The use according to claim 9, wherein said at least one activator is administered in combination with at least one adjuvant selected from a group consisting of at least one dipeptidyl peptidase-IV inhibitor, at least one insulin sensitizing agent, at least one biguanide, insulin, at least one insulin mimetic, at least one insulin secretagogues, at least one alpha glucosidase inhibitors, GLP-1, at least one GLP-1 analog and at least one GLP-1 receptor agonist.
